(19) United States
(12) Patent Application Publication
(10) Pub. No.: US 2004/0093030 A1
(43) Pub. Date: May 13, 2004

(54) Bone anchor and assembly

(76) Inventors: James E. Cox, Corcoran, MN (US); Brian P. Watschke, Eden Prairie, MN (US); Robert E. Lund, St. Michael, MN (US); Larry E. Waldack, Bloomington, MN (US); Richard M. Johnson, Minnetonka, MN (US)

Correspondence Address: John M. Haurykiewicz, FAEGRE & BENSON LLP, 2200 Wells Fargo Center, 90 South Seventh Street, Minneapolis, MN, 55402-3901, US

(21) Appl. No.: 10/335,070

(22) Filed: Dec. 31, 2002

Related U.S. Application Data
(60) Provisional application No. 60/421,747, filed on Oct. 28, 2002.

(51) Int. Cl. A61B 17/04

(52) U.S. Cl. 606/232

(57) ABSTRACT

A bone anchor assembly having a bone screw and a suture attachment, with the suture attachment threaded to the bone screw with a pair of mating threads that are of the opposite hand from the bone-engaging thread of the bone screw. Overtravel clearance is provided between the bone screw and the suture attachment to permit rotation of the suture attachment with respect to the bone screw at least in the same direction as the direction of engagement of the mating threads. The suture attachment can be an eye bolt or an eye nut. A pilot boss is received in a pilot bore in the engaging portions of the bone anchor and suture attachment. The bone anchor and suture attachment are anodized implant grade titanium material.

### CROSS-REFERENCE TO RELATED APPLICATION

This application claims priority of U.S. Provisional Patent Application No. 60/421,747, filed Oct. 28, 2002 and titled BONE ANCHOR AND ASSEMBLY. The entire contents of the above referenced provisional patent application are hereby incorporated herein by reference.

### BACKGROUND OF THE INVENTION

Orthopedic or soft tissue surgical procedures that utilize bone anchors are disclosed in U.S. Pat. Nos. 5,376,097, 5,572,342, 5,643,320, 5,741,282, and 5,980,558; percutaneous surgical procedures for treating incontinence are disclosed in U.S. Pat. Nos. 5,611,515, 5,836,314, 5,842,478, and 5,860,425; transvaginal surgical procedures for treating incontinence are disclosed in U.S. Pat. Nos. 5,972,000, 6,039,686, 6,053,935, 6,328,686, 6,322,492, and 6,334,446; and surgical procedures for treating airway obstructions, sleep apnea and snoring are disclosed in U.S. Pat. No. 5,988,171. Other incontinence procedures and/or surgical instruments are described in U.S. Pat. Nos. 6,241,736 and 6,319,272.

Bone anchors include bone screws, staples and bone tacks. U.S. Pat. Nos. 5,520,700, 5,674,247, 5,807,403 and 5,873,891 describe various bone anchors. Some bone anchors include a hole or eyelet for threading a suture to associate the suture with the bone anchor.

A variety of different surgical instruments have been developed to place a bone anchor. Some surgical instruments resemble a common screwdriver. Other surgical instruments are capable of placing a bone anchor at a remote region of the body, such as on a posterior portion of a patient's pubic bone or the patient's mandible.

Bone anchors are particularly useful as a component of a suprapubic sling procedure for stress urinary incontinence. Some procedures use a transvaginal approach and place a bone screw or tack on a posterior surface of the pubic bone.

The Vessica Sling Kits, available from Microvasive (affiliated with Boston Scientific and Scimed), utilize a percutaneous suprapubic approach to place a bone anchor on the top of the patient's pubic bone. The percutaneous suprapubic approach involves a relatively large abdominal incision. Complications associated with this procedure include osteitis pubis and osteomyelitis. See Appell R. A., The Use of Bone Anchoring in the Surgical Management of Female Stress Urinary Incontinence, World J. Urol. (1997); 15: 300-305; and Stram T. R., Ablove R. H., Lobel R. W., Infectious Complications of Vaginal Wall Suburethral Slings with Suprapubic Bone Anchors, Intl. Urogynecology J. (2000); 11 (Suppl. 1): abstract 015. Other surgical procedures utilixe a power drill to drill a hole for subsequent implantation of a bone anchor on top of a patient's pubic bone. After an abdominal incision is made, the surgeon prebores a hole in the bone with a drill, removes the drill, introduces a seating device, seats the bone anchor screw and then threads a suture. This procedure is somewhat cumbersome and it is believed that surgeons can lose access to the bored hole or seated bone screw. As a result, some surgeons need to drill additional holes and/or seat additional bone screws.

The Precision Twist™ Transvaginal Anchor System (available from Microvasive of Boston Scientific, of Natick, Mass.) includes a manually powered device for placing a bone screw in the posterior portion of a patient's pubic bone. The Precision Tack™ Transvaginal Anchor System (available from Microvasive) is used to place a tack on the posterior portion of a patient's pubic bone. The tack is manually inserted into the bone by pulling on the handle of the insertion device. The handle is elongate along an axis that is substantially aligned with the direction of pull.

The In-Fast™ Sling System is available from American Medical Systems, Inc. of Minnetonka, Minn. The In-Fast Sling System includes a single-use, battery-powered device that is capable of driving screws into bone. The inserter is shaped to place the screw on the posterior of the patient's pubic bone. The In-Fast inserter utilizes a rigid shaft and gear system to accomplish a ninety-degree bend. The In-Fast inserter receives a screw and associated suture in a screw mount. The device inserts a screw along a substantially straight insertion axis.

While the In-Fast inserter works well, it could manage the suture/bone anchor relationship even more conveniently and efficiently.

Placing a bone anchor in a remote region of a patient presents a particular challenge for a surgeon. Often, the intended location of the screw is surrounded by sensitive, vulnerable tissue such as arteries, nerves and veins. Such tissue can interfere with the proper operation of the bone anchor and/or associated suture during the use of the bone anchor inserter. The drawings show a bone screw assembly according to a preferred embodiment of the present invention, including various views of a preferred bone screw with a cavity for receiving a swivel and various views of the preferred swivel and bone screw assembly with the swivel.

### BRIEF DESCRIPTION OF THE DRAWINGS

FIG. 1 is a side view of a bone screw useful in the practice of the present invention.

FIG. 2 is an end view of the drive end of the bone screw of FIG. 1.

FIG. 3 is another view of the drive end of the bone screw shown in FIG. 2, showing the end of the thread on the bone screw.

FIG. 4 is a side sectional view of the bone screw of the present invention taken along line 4-4 of FIG. 3.

FIG. 5 is a side sectional view of the bone screw of the present invention taken along line 5-5 of FIG. 3.

FIG. 6 is an enlarged view of detail 6 showing a tip of the bone screw of FIG. 1.

FIG. 7 is a side view of a swivel useful with the bone screw of FIGS. 1-6 in the practice of the present invention.

FIG. 8 is a perspective view on a reduced scale of the swivel of FIG. 7.

FIG. 9 is a section view of the swivel taken along line 9-9 of FIG. 7.

FIG. 10 is a view of detail 10 of FIG. 9 showing cross sectional details of a threaded portion of the swivel of the present invention.

FIG. 11 is a transverse cross section taken along line 11-11 of FIG. 7.

FIG. 12 is a side view of the bone screw and swivel assembly of the present invention.

FIG. 13 is a section view of the bone screw and swivel assembly of the present invention taken along line 13-13 of FIG. 12.

### DETAILED DESCRIPTION OF THE INVENTION

The present invention affords convenient and efficient suture management and controlled, efficient operation of the bone anchor, the inserter and the associated suture. More particularly, the present invention provides for a swivel to allow the suture attachment to rotate relative to the bone anchor such that excessive twisting of the suture is avoided when the bone anchor is rotated as it is installed. This aspect of the present invention is accomplished by having an interengaging pair of threads between the suture attachment and the bone anchor, with the interengaging threads of the suture attachment and the bone anchor having an opposite hand from a bone engaging thread of the bone anchor and having a sufficient amount of overtravel such that the threads in the swivel can advance sufficiently in the mating threads in the bone anchor until the threads disengage from each other, while still maintaining an axial retention between the swivel and bone anchor because of the capture of the swivel threads by the threads of the bone anchor.

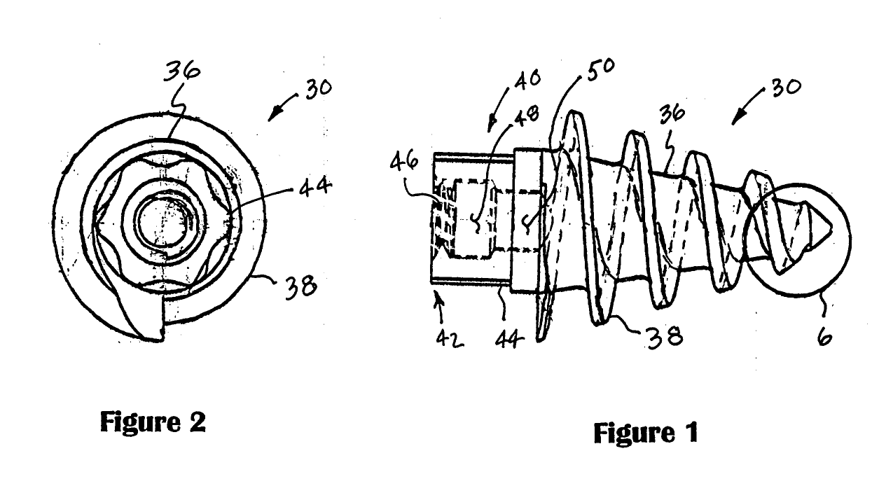

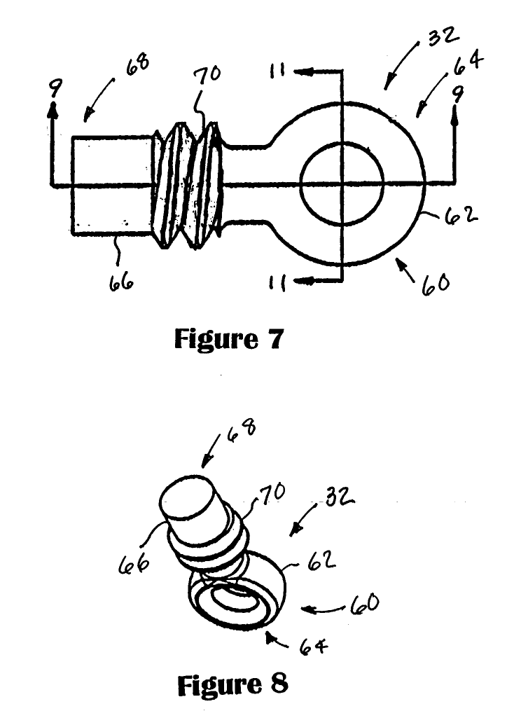

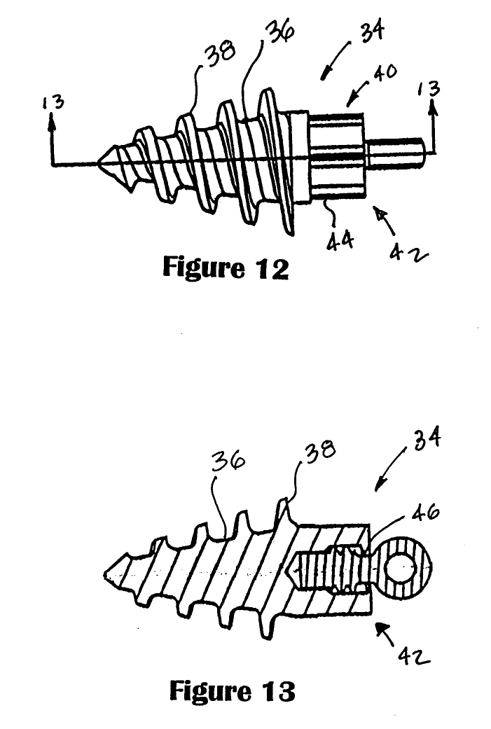

Referring to the Figures, and most particularly, to FIGS. 1 and 7, a bone anchor 30 and a suture attachment or swivel 32 may be seen. Referring also to FIGS. 12 and 13, the assembly 34 made up of anchor 30 and suture attachment 32 may be seen in an assembled form.

As used herein, a “screw thread” or “thread” is a substantially continuous, radially-projecting helical ridge. An “internal thread” has the ridge projecting radially inwardly, and an “external thread” has the ridge projecting radially outwardly. A thread is a “right hand thread” if, when viewed axially, it winds in a clockwise and receding direction. Also as used herein, a thread is a “left hand thread” if, when viewed axially, it winds in a counterclockwise and receding direction. A “bone screw” is a mechanical fastener which uses a screw thread for attachment to the hard, calcified tissue of the skeleton of a vertebrate animal.

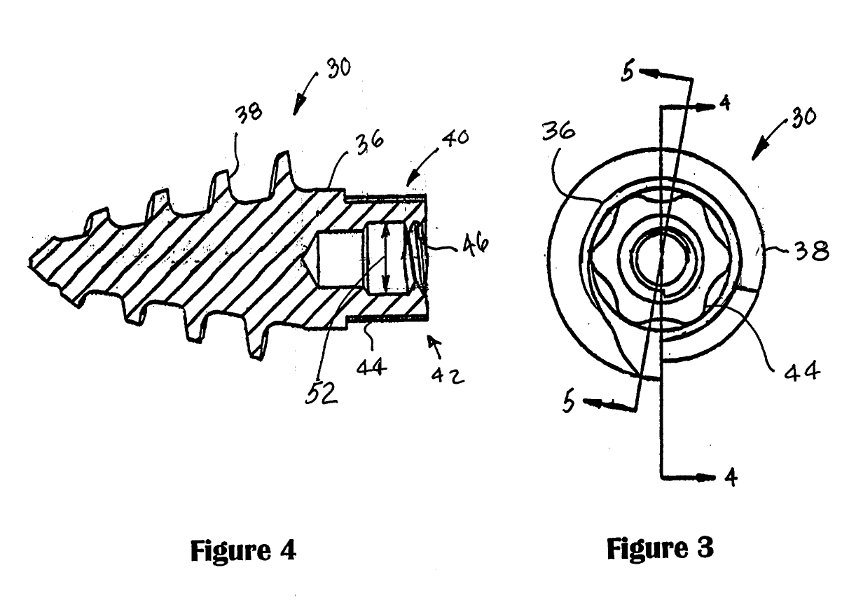

The bone anchor 30 is a bone screw with an external bone engaging thread 38. As shown, the bone screw is conical, and the bone engaging thread is conical, although it is to be understood that other forms (e.g., cylindrical) of bone screws and bone engaging threads may be used while still remaining within the present invention. The bone engaging thread 38 shown is a right hand thread. Anchor 30 also has a drive connection 40 located at a proximal end 42 of the screw 36. Drive connection 40 is preferably a spline 44 which may be seen most clearly in FIGS. 2, 3 and 12. Alternatively, drive connection 40 can take another form of non-circular or non-concentric geometry to provide a connection to the bone anchor capable of transmitting torque to install the bone anchor.

As shown most clearly in FIGS. 1, 4, 5 and 13, anchor 30 has an internal left hand thread portion 46. Axially interior of threaded portion or thread 46 is a clearance bore 48 and interior of bore 48 is a pilot bore 50. Preferably clearance bore 48 has an internal diameter 52 equal to or greater than the maximum thread diameter of threaded portion 46. Pilot bore 50 preferably has a reduced diameter 54 which is less than diameter 52. Thread portion 46 preferably has only one revolution of its thread. Thread 46 is a first mating thread and is preferably a 1.00 UNM L.H. internal thread.

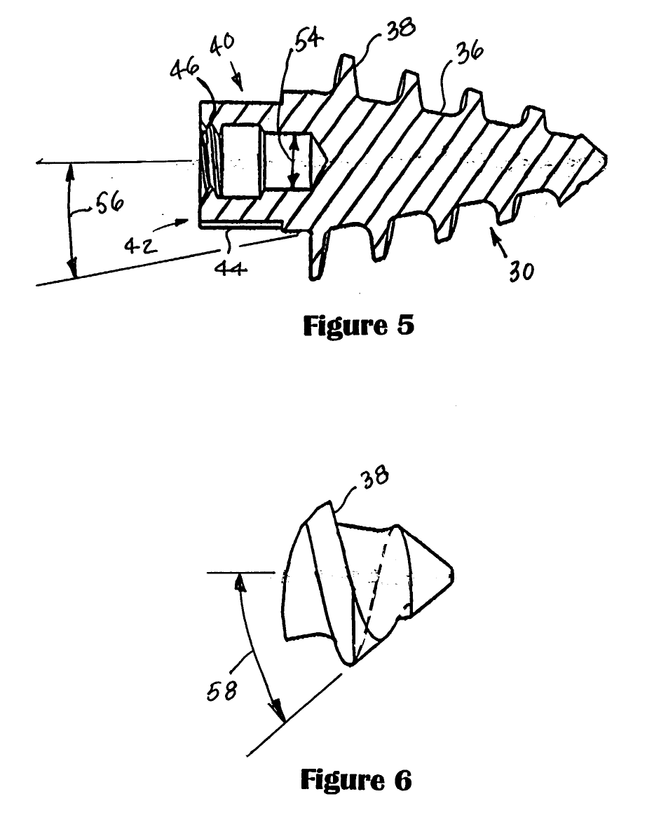

As may be seen in FIGS. 5 and 6, anchor 30 preferably has a cone angle 56 of about 10.5 degrees and the tip 58 of anchor 30 preferably has a cone angle 58 of about 40 degrees.

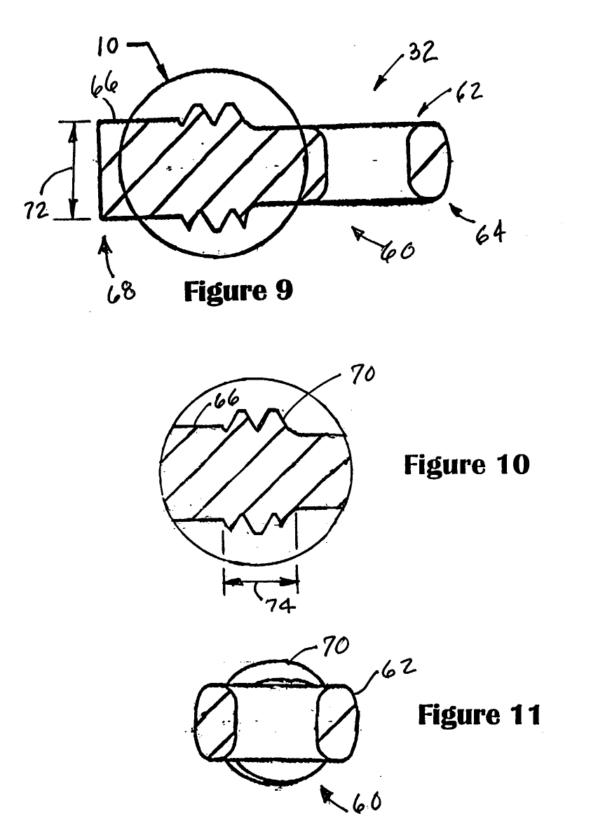

Referring now most particularly to FIGS. 7 through 11, the suture attachment 32 is preferably an eye bolt 60, having an eye 62 at a proximal end 64 and a pilot boss 66 at a distal end 68. Eye bolt 60 also has a thread portion or thread 70 intermediate the proximal and distal ends. Thread portion 70 is a second mating thread for interengagement with the first mating thread 46 of the anchor 30. Eye 62 preferably has an outside diameter sufficiently small to allow a mating splined installation tool (not shown) to be received over eye 62 to engage spline 44. Pilot boss 64 preferably has a diameter 72 sized to provide a clearance fit with the diameter 54 of pilot bore 50. Thread portion 70 is preferably a 1.00 UNM L.H. external thread and preferably extends for an axial distance 74 greater than one revolution of the thread.

As an optional feature or step in the practice of the present invention, at least one of the mating threads 46, 70 may be crimped after assembly to prevent disassembly of the bone anchor and suture attachment. Preferably a collet (not shown) is placed over the proximal end of the spline 44 and thread 46 is crimped radially inwardly, to provide interference and to prevent unthreading engagement between threaded portion 46 and threaded portion 70, while still permitting relative rotation between the suture attachment and bone anchor after crimping.

The parts making up the bone anchor and suture attachment of the present invention are preferably made of an industry standard implant grade titanium material, identified as Ti6AL4VELI. The parts may also be anodized, to reduce friction.

This invention is not to be taken as limited to all of the details thereof as modifications and variations thereof may be made without departing from the spirit or scope of the invention. For example, and not by way of limitation, the bone-engaging thread may be a left hand thread, provided that the mating threads connecting the bone anchor and suture attachment together are right hand threads.

For another example, and not by way of limitation, the bone anchor may be provided with a threaded stud at the proximal end thereof and the eye bolt replaced by an eye nut with sufficient overtravel relief between the threads of the stud and the body of the bone anchor to permit free rotation of the eye nut on the stud once the eye nut has been fully threaded on the stud. Such a reversal of parts or portions of parts is still within the scope of the present invention. It is also to be understood that the present invention may be practiced with various other bone screw configurations, such as a bone screw with a drilling flute at the distal tip of the bone screw.

What is claimed is:

1. An improved bone anchor and suture attachment assembly of the type having a bone screw with an external bone-engaging thread having one of right hand thread and left hand thread for engaging a bone and a drive connection and a suture attachment both located at a proximal end of the screw, the improvement in combination with the bone anchor and suture attachment comprising:
a. a pair of mating threads having the other of the right hand thread and left hand thread, with one of the pair of mating threads on the bone screw and the other of the mating threads on the suture attachment; and
b. an overtravel relief for the mating threads retaining the suture attachment to the bone anchor while permitting rotation between the bone anchor and suture attachment.

2. The assembly of claim 1 wherein the external bone-engaging thread is a right hand thread and the pair of mating threads are left hand threads.

3. The assembly of claim 1 wherein the bone screw is conical.

4. The assembly of claim 3 wherein the bone engaging thread is conical.

5. The assembly of claim 1 wherein the one of the mating threads on the bone screw is an internal thread.

6. The assembly of claim 5 wherein the one of the mating threads has only one revolution of the internal thread.

7. The assembly of claim 1 wherein the suture attachment comprises an eye for receiving a suture.

8. The assembly of claim 7 wherein the suture attachment is an eye bolt.

9. The assembly of claim 1 wherein the overtravel relief comprises an axial clearance between the bone anchor and the suture attachment sufficient to permit rotation therebetween and to provide axial retention between the bone anchor and the suture attachment once the mating threads of the bone anchor and suture attachment have been moved past a threaded interengagement.

10. The assembly of claim 9 wherein at least one of the mating threads is crimped in an amount sufficient to prevent re-engagement of the mating threads after the mating threads have been moved past the threaded interengagement while still permitting rotation between the bone anchor and the suture attachment.

11. The assembly of claim 1 wherein the bone anchor and suture attachment are formed of a titanium material.

12. The assembly of claim 11 wherein the bone anchor and suture attachment are formed of an implant grade titanium material.

13. The assembly of claim 12 wherein the bone anchor and suture attachment are formed of material having a designation Ti6AL4VELI.

14. The assembly of claim 13 wherein the bone anchor and suture attachment are anodized.

15. The assembly of claim 1 wherein the suture attachment further comprises a pilot boss and the bone anchor further comprises a pilot bore sized to receive the pilot boss in a clearance fit therebetween.

16. A method of forming a swivelling suture attachment to a bone anchor comprising:
a. forming a bone anchor with a proximal end and a distal end and having
i. an external bone-engaging thread having one of right hand thread and left hand thread for engaging a bone and extending toward the distal end of the bone anchor, and
ii. a drive connection at the proximal end of the bone anchor, and
iii. a first mating thread having one of an internal and external thread and having the other of the right hand thread and left hand thread, the first mating thread at the proximal end of the bone anchor;
b. forming a suture attachment with a proximal end and a distal end and having
i. an eye for receiving a suture at the proximal end of the suture attachment, and
ii. a second mating thread having the other of the internal and external thread and the other of the right hand thread and left hand thread, the second mating thread distal of the eye of the suture attachment;
c. attaching the suture attachment to the bone anchor by interengaging the first and second mating threads; and
d. advancing the first and second mating threads past interengagement and permitting relative rotation between the bone anchor and suture attachment while preventing axial separation of the suture attachment from the bone anchor.

17. The method of claim 16 wherein the relative rotation of the bone anchor and suture attachment is in the same direction as a direction for initially interengaging the first and second mating threads.

18. The method of claim 16 further comprising an additional step:
e. crimping at least one of the first and second mating threads after the first and second threads have been advanced past interengagement such that the first and second mating threads are thereafter not able to be interengaged for unthreading.

19. An improved bone anchor and suture attachment assembly formed by the process of:
a. threading a suture attachment to a bone anchor by threadingly engaging a pair of mating threads where one of the pair of threads is on the bone anchor and the other of the pair of mating threads is on the suture attachment and each of the pair of mating threads has the same one of a left hand thread and a right hand thread, with the bone anchor having a bone-engaging thread having the opposite pair of mating threads having the opposite of the handedness of a bone-engaging thread on the bone anchor; and
b. advancing the mating threads of the suture attachment and bone anchor past interengagement and permitting relative rotation between the bone anchor and suture attachment while maintaining axial attachment of the suture attachment to the bone anchor.

20. The improved bone anchor and suture attachment assembly formed by the process of claim 19 wherein the process further comprises:
c. crimping at least one of the pair of mating threads after step b. to prevent reengagement of the mating threads while still permitting relative rotation between the bone anchor and suture attachment.

21. The improved bone anchor and suture attachment assembly formed by the process of claim 19 wherein the process further comprises an additional step a0. performed before step a. as follows:
a0. anodizing a bone anchor and a suture attachment.
